(19) United States
(12) Patent Application Publication
(10) Pub. No.: US 2003/0204264 A1
(43) Pub. Date: Oct. 30, 2003

(54) Prosthetic revision knee system

(76) Inventors: David J. Stumpo, Venetia, PA (US); Donald A. Stevens, Spring Church, PA (US)

Correspondence Address: KIRKPATRICK & LOCKHART LLP, 535 SMITHFIELD STREET, PITTSBURGH, PA, 15222, US

(21) Appl. No.: 10/441,577

(22) Filed: May 20, 2003

Related U.S. Application Data
(63) Continuation of application No. 09/792,504, filed on Feb. 23, 2001, now abandoned.

(51) Int. Cl. A61F 2/38

(52) U.S. Cl. 623/20.36

(57) ABSTRACT

The invention is in the field of orthopedic implants. More specifically, the invention relates to a revision knee implant. The implant of the invention includes a femoral component, a bushing and a locking member and is configured for positioning, in use, in the natural knee joint. The implant also may include a femoral stem. The femoral component has spaced apart condylar portions defining an intercondylar region. The intercondylar region includes elongate engagement rails that extend in the anterior-posterior direction. The bushing slides along the length of the engagement rails. The locking member locks the bushing in a desired position in the anterior-posterior direction of the engagement surface. The femoral stem is configured for insertion, in use, into the femoral canal, and is also configured for secure attachment to the bushing at an angle in substantial alignment with the valgus angle of the patient. When a femoral stem is in position in the bushing, the locking member is captured between the femoral component and the femoral stem.

### BACKGROUND OF THE INVENTION

Knee joint arthroplasty is a common surgical procedure by which a failing natural knee is replaced with a prosthetic knee. The natural knee may fail due to trauma, such as a car accident, or due to degenerative disease, such as arthritis.

There are two types of prosthetic knee joints: a primary knee and a revision knee. A primary knee prosthesis is used when failure of the natural knee is not severe. For example, a primary knee prosthesis can be used when the ligaments in the natural knee are still intact. In addition, the prosthesis may not require a femoral stem. On the other hand, a revision knee prosthesis is used for more severe failure of the natural knee or failure of the primary knee prosthesis. A revision knee prosthesis typically requires a femoral stem.

In patients who require a revision knee system, there is a lot of bone loss, and therefore, a lack of normal bony reference points or landmarks for properly aligning the implant. In these cases, surgeons use the intramedullary canal of the femur as the landmark for positioning the prosthesis. If the femoral stem of the implant is not properly aligned with respect to the intramedullary canal in the anterior-posterior direction, there will be a gap between the natural femur and the femoral component, at the anterior or the posterior end.

Conventional knee prostheses include a tibial component, a femoral component, a femoral stem and a patellar component. The tibial component is usually a T-like structure that is attached to the tibia. The tibial component has a surface on top of the T that is designed to articulate with the femoral component. The femoral component typically includes a pair of spaced apart condylar portions that articulate with the tibial component. In addition, the femoral component usually has an intercondylar surface, sometimes referred to as the intercondylar box, located between the two condylar portions. The intercondylar surface defines a box-like region that is either open or closed. The femoral stem is typically connected to the intercondylar surface of the femoral component by means of a bolt that passes through a bore in the intercondylar surface. The femoral stem is, in use, inserted into a reamed intramedullary canal of the femur. The patellar component articulates with the anterior surface of the femoral component.

The aforementioned components come in various sizes and shapes to compatibly match the anatomical constraints of a variety of individual patients. In addition, to further match a particular patient's anatomical constraints, the femoral stem of the prosthesis may be set at an angle, from lateral to medial, to match a patient's valgus angle. The valgus angle is the angle between the center line of the femur and an imaginary vertical line extending from the distal femur to the center of the femoral head.

Despite the above anatomical accommodations, conventional knee prostheses have some notable shortcomings. For example, the anterior/posterior position of the femoral stem is either not adjustable or only adjustable in limited increments, leading in both cases to poor alignment of the femoral prosthesis with respect to the intramedullary canal of the femur. If the prosthesis does not fit correctly anatomically, deterioration can occur. In one knee system, incremental anterior-posterior positioning is provided by means of three interchangeable screw heads that permit limited variation of the anterior/posterior position from the norm (e.g., 0 mm) by increments of +3 and −3 mm.

In some knee prostheses, the femoral stem is attached to the femoral component by means of a nut and bolt. If the bolt becomes loosened, it can migrate into the knee joint, leading to injury to the surrounding tissue and damage to or interference with the prosthesis. In addition, a nut and bolt system can cause additional problems by introducing particulate matter into the knee joint. This could cause an infection or lead to an inflammatory immune response.

There is a need for a prosthetic revision knee system wherein the anterior/posterior position of the femoral stem can be infinitely adjusted to suit a wide range of patient anatomies. In addition, there is a need for a knee system that permits valgus offset of the femoral stem, so as to accommodate the anatomical constraints of a variety of patients.

There is a further need for a prosthetic revision knee system that utilizes a locking mechanism that will not migrate into the knee joint.

### BRIEF SUMMARY OF THE INVENTION

The present invention provides a prosthetic revision knee system that is used to replace a severely damaged natural knee or an existing knee prosthesis. Revision knee systems are commonly required in patients with severe trauma or degenerative disease of the knee.

The implant of the present invention is configured for positioning, in use, in the natural knee joint and comprises a femoral component, a bushing and a locking member. The femoral component has spaced apart condylar portions defining an intercondylar region therebetween, the intercondylar region having an elongate engagement surface extending in the anterior-posterior direction. The bushing is configured for sliding engagement with the engagement surface of the intercondylar region for movement in the anterior-posterior direction. The locking member locks the bushing in a desired position along the anterior-posterior direction on the engagement surface.

The bushing preferably has a base portion, an upper portion and a bore therethrough. The base portion preferably has mating surfaces for sliding engagement along the engagement surface of the intercondylar region, and the bore preferably receives the locking member. The mating surfaces of the base portion of the bushing are designed to mate with the complementary engagement surfaces of the femoral component so that the bushing may be securely engaged into the proper anterior/posterior location on the femoral component.

After the bushing has been positioned into the desired anterior/posterior location on the femoral component, the locking member is used to lock the position of the bushing on the femoral component. This is preferably accomplished by inserting a set screw into he bore of the bushing and tightening the set screw with a device, such as a torque wrench. The preferred embodiment of the set screw has a distal surface that extends slightly beyond the base portion of the bushing so as to form a pressure fit against the femoral component.

The bushing, and preferably the upper portion of the bushing, may be canted from the lateral to the medial direction at an acute angle. The bushing can have any one of a number of different canting angles in order to substantially match the valgus angle of a patient recipient.

The implant of the present invention may itself include a femoral stem, or be configured for attachment to a femoral stem. The bushing preferably includes a minor bore for capture of the locking member in the base portion and a major bore for securing the femoral stem in the upper portion. The femoral stem is configured for insertion, in use, into the patient's femoral canal. The upper portion of the bushing is configured for secure attachment to the femoral stem. The locking member is captured in the base of the bushing between the femoral component and the femoral stem, thereby preventing the locking member from being dislodged.

The implant of the present invention may also include a tibial component and/or a patellar component. Any compatible tibial component and/or patellar component may be used with the implant of the present invention.

Other details, objects and advantages of the present invention will become apparent with the following brief description of the several views of the drawings and the detailed description of the invention.

### BRIEF DESCRIPTION OF THE SEVERAL VIEWS OF THE DRAWINGS

For the purpose of illustrating the preferred embodiments and methods of the prosthetic revision knee system of the present invention and not for limiting the same, reference is made to the drawings in which:

FIG. 1 is a posterior view of an embodiment of a fully assembled prosthetic revision knee system implant of the present invention.

FIG. 2A is a top isometric view of an embodiment of a femoral component. FIG. 2B is a side isometric view of an embodiment of a femoral component.

FIG. 3A is a front elevation view of an embodiment of a bushing. FIG. 3B is an isometric view of the same embodiment of the bushing as in FIG. 3A.

FIGS. 4A and 4B are isometric views of an embodiment of a bushing in two different positions on the femoral component. FIG. 4C is a posterior view of the embodiment of the bushing and femoral component shown in FIGS. 4A and 4B, showing medial/lateral offset at an acute angle to match a patient's valgus angle.

FIG. 5A is an exploded view of an embodiment of a bushing and an embodiment of a locking member.

FIG. 5B is an isometric bottom view of the embodiment of the bushing and locking member shown in FIG. 5A with the locking member inserted in a bore of the bushing.

FIG. 6A is an isometric view of an assembled femoral component, bushing, femoral stem and locking member (hidden). FIG. 6B is a posterior view of the assembled components of FIG. 6A.

FIG. 7A is a front elevation view of an alternative embodiment of a bushing showing dovetailed engagement surfaces. FIG. 7B is an isometric view of an alternative embodiment of a femoral component showing dovetailed engagement surfaces.

### DETAILED DESCRIPTION OF THE INVENTION

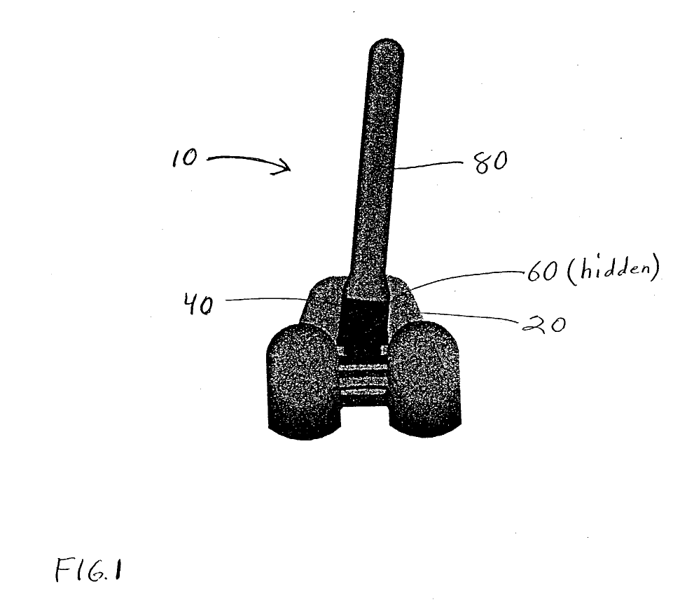

The present invention provides a prosthetic revision knee system including an implant 10. FIGS. 1 through 7 illustrate various embodiments of the prosthetic revision knee system. Referring to FIG. 1, an embodiment of the implant 10 of the present invention includes a femoral component 20, a bushing 40 and a locking member 60. The knee system preferably also includes a femoral stem 80.

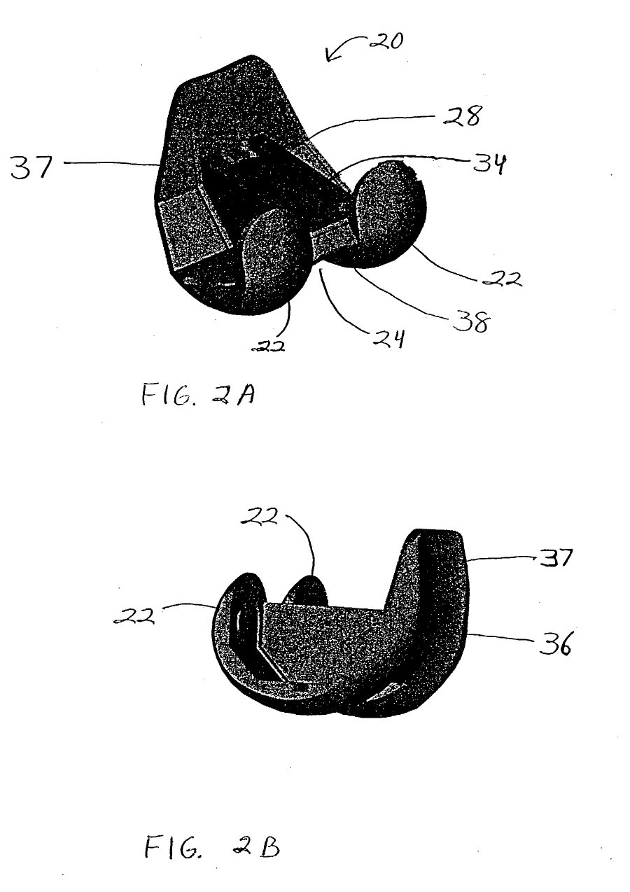

An embodiment of a femoral component 20 is illustrated in FIG. 2A. The femoral component 20 is configured for positioning, in use, in a natural knee joint (not shown). The configuration of the femoral component 20 can have a variety of shapes and sizes. However, the femoral component 20 is preferably similar in shape to a natural knee so that it can function as a natural knee would. Preferably, the femoral component 20 has a pair of spaced-apart condylar portions 22 that define an intercondylar region 24. The condylar portions 22 allow the femoral component 20 to articulate with a tibial component (not shown). In addition, the femoral component 20 is preferably symmetrical so that it can be used in either the right or left knee. However, the femoral component 20 is not required to be symmetrical. Furthermore, the femoral component 20 preferably has a patellar groove 36 on its anterior surface 37 so that it can accommodate either a natural or artificial patella (not shown), as illustrated in FIG. 2B.

Referring again to FIG. 2A, the femoral component 20 of the present invention has an intercondylar region 24. The intercondylar region 24 can have almost any length or width, within the limitations of the femoral component 20 and within anatomical limitations of the patient. Preferably, the intercondylar region 24 spans the length of the femoral component 20 in the anterior and the posterior direction. In addition, the intercondylar region 24 preferably has a width in the medial/lateral direction of at least 14 mm in order to provide sufficient medial/lateral stability to the bushing 40.

The intercondylar region 24 has an elongate engagement surface 28 extending in the anterior-posterior direction. The engagement surface 28 is a closed. The engagement surface 28 can be any shape or size of surface (within the limitations of the femoral component 20 and within anatomical limitations of the patient) that would allow for complementary engagement with another surface. Preferably, the engagement surface 28 includes opposing rails 34. The engagement surface 28 preferably spans the entire length of the intercondylar region 24 to maximize the range of anterior/posterior offset of the femoral stem. However, the engagement surface 28 can span a shorter length of the intercondylar region 24 while still offering a range of anterior-posterior positioning not found in the prior art revision knee systems.

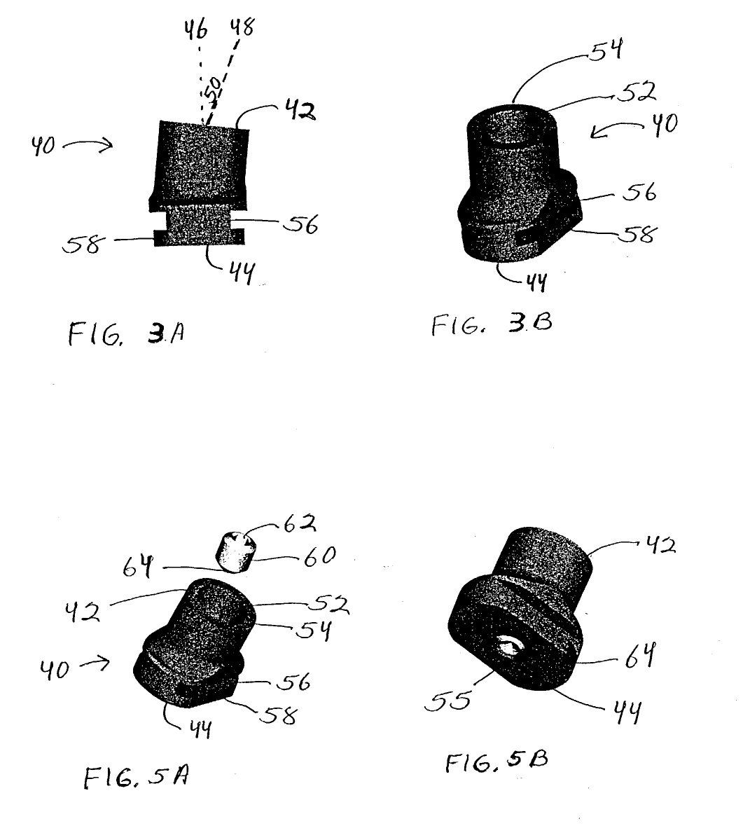

An embodiment of a bushing 40 is illustrated in FIGS. 3A and 3B. The bushing 40 is configured for sliding engagement along the engagement surface 28 of the intercondylar region 24. The bushing 40 can be shaped in a variety of configurations. Exemplary configurations for the bushing 40 are shown in any of FIGS. 3A, 3B, 5A, 5B or 7A. The bushing 40 is preferably symmetrical so that it can be used in either the right or left knee. However, the bushing 40 does not have to be symmetrical and may be right or left knee specific. If the bushing 40 is symmetrical, a surgeon only has to rotate it 180° to have the bushing 40 match the valgus angle for either the right or left knee.

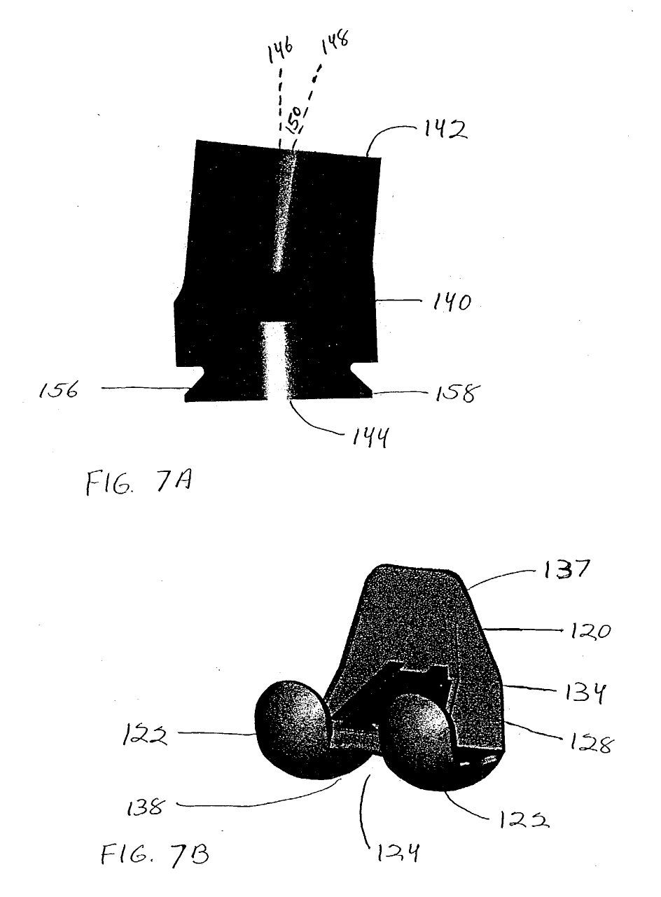

The bushing 40 preferably has an upper portion 42 and a base portion 44. The base portion 44 of the bushing 40 includes mating surfaces 56. The mating surfaces 56 can be any shape or size of surface (within the limitations of the bushing 40 and within anatomical limitations of the patient) that would allow for engagement with the engagement surface 28 of the intercondylar region 24 of a femoral component 20. Preferably, the mating surfaces 56 form rectangular grooves 58 (FIGS. 3A and 3B). More preferably, the mating surfaces 156 are in the form of dovetails 158 (FIG. 7A). The base portion 44 of the bushing 40 may, but need not, be the same length or width as the upper portion 42.

The bushing 40 has a bore 52 therethrough. The bore 52 extends from the upper portion 42 to the base portion 44 of the bushing 40. The width of the bore 52 is wide enough to allow a locking member 60 to pass through the bore 52 from the upper portion 42 to the base portion 44, even if the bushing 40 is canted at an acute angle. However, the locking member 60 should fit snugly in the base portion 44 of the bushing 40. This means that the width of the bore 52 need not be constant. For example, the bore 52 may be wider at the upper portion 42 than at the base portion 44. In addition, the bore 52 may be threaded for receiving the locking member 60.

The bore 52 preferably has a minor bore 55 in the base portion 44 and a major bore 54 in the upper portion 42. The minor bore 55 is for receiving the locking member 60. The major bore 54 is configured for receiving a femoral stem 80 in the implant 10. Preferably, the major bore 54 and the minor bore 55 are both threaded.

The bushing 40 is preferably canted in the medial/lateral direction at an acute angle so as to have a canting angle 50. The femoral component 20 is configured such that a plane of symmetry is defined between the condylar portions 22 in the anterior-posterior direction. The bushing 40 has a base portion 44 including mating surfaces 56 that define a central axis 46 lying in the plane of symmetry, and an upper portion 42 that defines a central axis 48 offset from said plane of symmetry at an acute angle, the canting angle 50. The canting angle 50 is designed to match or substantially match a patient's valgus angle. Preferably, the canting angle 50 is in the range of 0 to about 9 degrees. More preferably, the canting angle 50 is one of 0, 3, 5 or 7 degrees. In a commercial embodiment of the invention, bushings of a range of canting angles would be provided.

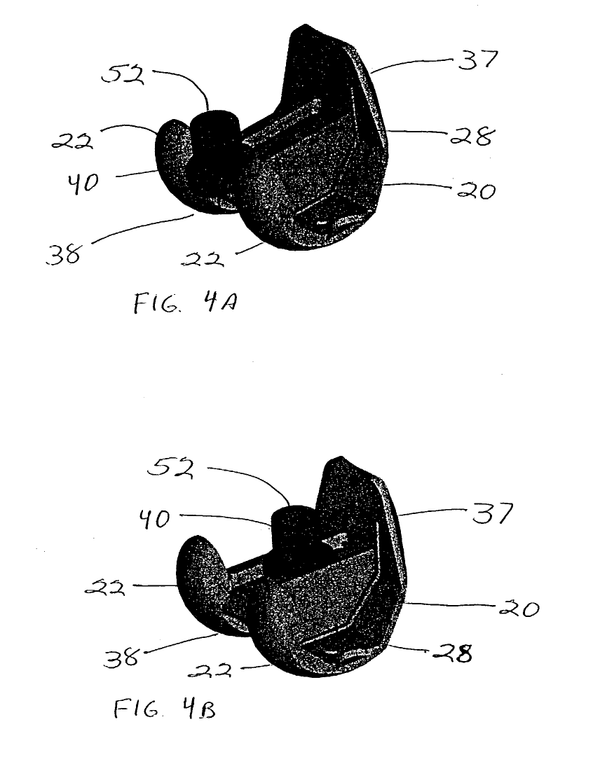

FIGS. 4A, 4B and 4C illustrate the engagement of an embodiment of a bushing 40 to an embodiment of a femoral component 20. FIG. 4A shows the bushing 40 being inserted into a sliding engagement with the femoral component 20. The bushing 40 is slideably inserted from the posterior surface 38 of the femoral component 20 toward the anterior surface 37, as shown in FIGS. 4A and 4B. The engagement surface 28 in the intercondylar region 24 of the femoral component 20 mates with the mating surfaces 56 of a bushing 40 so as to allow the mating surfaces 56 to slide across the engagement surface 28, as shown in FIGS. 4A and 4B. Preferably, the engagement surface 28 comprises opposing rails 34 that are complementary to the mating surfaces 56, and the mating surfaces 56 comprise rectangular grooves 58, as illustrated in FIG. 4C. More preferably, the engagement surface 128 comprises opposing rails 134 that are complementary to the mating surfaces 156, and the mating surfaces 156 form dovetails 158, as illustrated in FIGS. 7A and 7B. This sliding engagement enables a surgeon to match the anterior/posterior offset of the implant 10 across a wide range and an infinite number of positions along the length of the rails 134, thus allowing for a more anatomically correct fit. In addition, the sliding engagement gives the bushing 40 good medial/lateral stability.

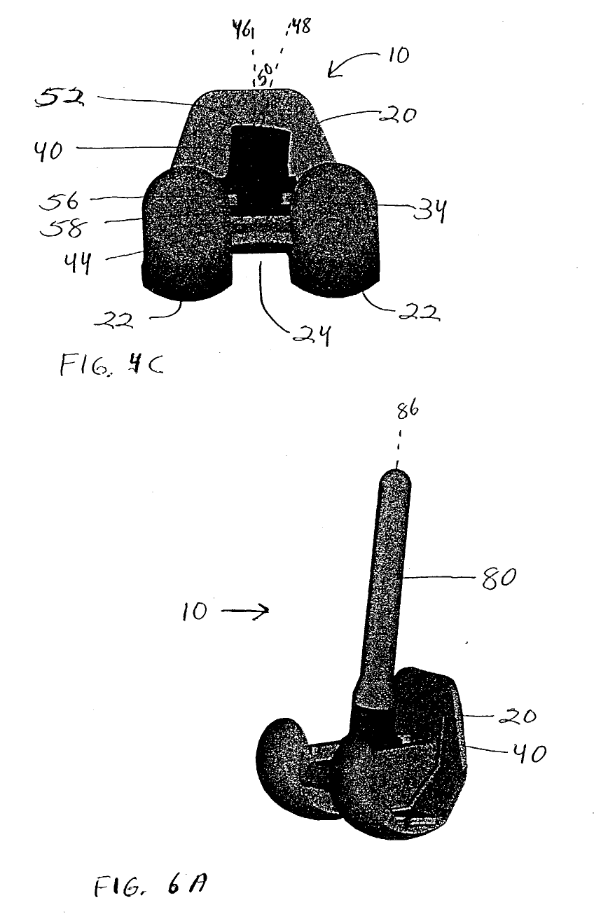

After the correct anterior/posterior position of the bushing 40 is located on the femoral component 20, the bushing 40 is locked into place on the femoral component 20 by means of a locking member 60. In order to lock the bushing 40 into place, the locking member 60 is inserted into a bore 52 of the bushing 40, as shown in FIG. 5A. The locking member 60 then travels from the upper portion 42 to the base portion 44 of the bushing 40, as shown in FIG. 5B. Once at the base portion 44 of the bushing 40, the locking member 60 may be tightened by a device, such as a torque wrench, in order to lock the position of the bushing 40 on the femoral component 20. The locking member 60 may be threaded on the outside so as to provide a mating surface with a complementary surface of the minor bore 55. The inside of the locking member or a surface on the locking member is configured for releasable engagement with the tightening device. A commercial embodiment of the prosthesis would preferably be factory assembled so that the locking member 60 is preplaced in minor bore 55. When the surgeon places the bushing 40 in the desired anterior-posterior location, the already in-place locking member 60 would then be tightened to lock the bushing in the desire position.

Still referring to FIGS. 5A and 5B, the locking member is preferably a set screw. The set screw is preferably rounded and has both a proximal end 62 and a distal end 64. The proximal end 62 has a surface that allows the set screw to be tightened intraoperatively by means of a device, such as a torque wrench. The distal end 64 of the set screw has a surface that, in use, extends beyond the base portion 44 of the bushing 40 so that upon tightening, it is able to contact the femoral component 20 and form a pressure fitting against the femoral component 20 so as to lock the position of the bushing 40 against the femoral component 20. The pressure fitting is generated by the torque applied to the set screw as it is being tightened. In addition, the set screw may be threaded on the outside so as to provide a mating surface with a complementary surface of the minor bore 55.

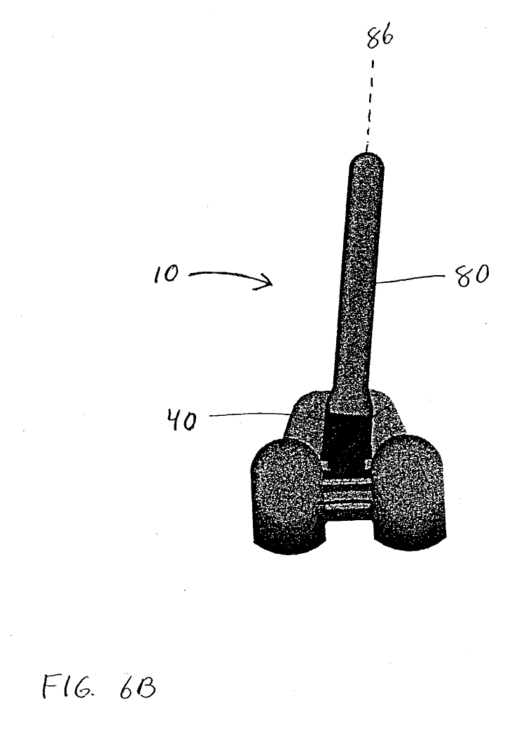

An embodiment of the implant 10 including a femoral stem 80 is illustrated in FIGS. 6A and 6B. The femoral stem 80 is configured for insertion, in use, into the intramedullary canal of a femur (not shown). The femoral stem 80 may be linear or may be curved along its longitudinal axis 86, depending upon the anatomy of the patient's femoral canal. In addition, the cross-sectional configuration of the femoral stem 80 can have any one of a number of shapes, although, a circular cross-sectional configuration is preferred. Furthermore, the femoral stem 80 can have one of a variety of different combinations of length and width.

The femoral stem 80 is configured for positioning at a distal end thereof in the bore 52 of a bushing 40. As stated previously, the femoral component 20 is configured such that a plane of symmetry is defined between the condylar portions 22 in the anterior-posterior direction. The bushing 40 has a base portion 44 including mating surfaces 56 that define a central axis 46 lying in the plane of symmetry, and an upper portion 42 defining a central axis 48 offset from the plane of symmetry at an acute angle. The acute angle is the canting angle 50. The femoral stem 80 is configured for insertion, in use, into the femoral canal, and the upper portion 42 of the bushing 40 is configured for secure attachment to the femoral stem 80 so as to align the femoral stem 80 at the acute angle, the canting angle 50, along the central axis 48 of the upper portion of the bushing 40. The combination of the anterior-posterior positioning freedom and the range of valgus angle positions offered by the present invention permits the axis of the femoral stem 80 to be properly aligned within the femoral canal in both the anterior-posterior position and along the patient's valgus angle.

Some known prostheses allow limited incremental anterior-posterior positioning, but do not provide the infinite variation along the length of the engagement surface provided by the implant 10 of the present invention. The implant 10 of the present invention permits the bushing, and therefore, the femoral stem when attached, to be moved through and locked into any position along the anterior posterior length of the intercondylar region. The femoral prosthesis is therefore in intimate contact with the femur, leaving no gaps between the femur and the anterior or posterior ends of the prosthesis.

The bore 52 in bushing 40 preferably includes a minor bore 55 in the base portion 44 of the bushing 40 and a major bore 54 in the upper portion 42 of the bushing 40. The minor bore 55 is configured to receive the locking member 60 while the major bore 54 is configured to receive the femoral stem 80. The major bore 54 may be threaded so as to receive a complementary surface of the femoral stem 80.

An advantage of attaching a femoral stem 80 to the implant 10 is that it gives the implant 10 added stability in the knee joint. In addition, the femoral stem 80 captures the locking member 60. The locking member 60 is captured because it is located inside the bushing 40, between the femoral component 20 and the femoral stem 80. Therefore, the locking member 60 cannot become dislodged from its set position. Preferably, the femoral stem 80 is engaged with the major bore 54 to a point where the femoral stem 80 sits on top of the proximal end 62 of the locking member 60.

An alternative embodiment of the engagement surfaces of the bushing and femoral component are illustrated in FIGS. 7A and 7B. The bushing 140 is similar to the previously described bushing 40, except that the mating surfaces 156 are in the form of dovetails 158. The femoral component 120 is similar to the previously described femoral component 20, except that the opposing rails 134 are in the form of dovetails complementary to the dovetails 158 of the mating surfaces 156 of the bushing 140. Dovetails 158 are preferred over rectangular grooves 58 because the dovetail shape enables the base portion 144 of the bushing 140 to be compressed. In addition, the dovetails allow for a more stable construct because they give greater medial/lateral stability.

Those skilled in the art will appreciate that the implant 10 can be made in a variety of sizes that fall within anatomical constraints of the patient population. In order to accommodate patient differences, the various modular components of the implant 10 of the present invention can be made in a variety of sizes and shapes that are interchangeable with the other components.

The components of the implant 10 of the present invention can be made from any biocompatible material or materials. Suitable materials include cobalt chrome, titanium and stainless steel. Preferably, femoral components, 20 and 120, are made of titanium or cobalt chrome. The alternative embodiments of the bushing, 40 and 140, are also preferably made of titanium or cobalt chrome. The femoral stem 80 is preferably made of titanium. Lastly, the locking member 60 is preferably made of titanium or cobalt chrome. Although not required, the above materials may be coated with a biocompatible material.

The implant 10 of the present invention thus solves many of the problems encountered by prior knee prostheses. Those of ordinary skill in the art will appreciate that various changes in the details, methods, materials and arrangement of parts which have been herein described and illustrated in order to explain the nature of the invention may be made by the skilled artisan within the principle and scope of the invention as expressed in the appended claims.

What is claimed is:

1. An implant for replacing at least a portion of a natural knee comprising:
a femoral component having spaced apart condylar portions defining an intercondylar region therebetween, said intercondylar region having an elongate engagement surface extending in an anterior-posterior direction;
a bushing configured for sliding engagement with said engagement surface of said intercondylar region for movement in said anterior-posterior direction; and
a locking member for locking said bushing in a desired position in said anterior-posterior direction on said engagement surface.

2. The implant recited in claim 1 wherein said engagement surface includes opposing rails and said bushing has complementary mating surfaces configured to slide along said rails.

3. The implant recited in claim 2 wherein said mating surfaces of said bushing are dovetailed.

4. The implant recited in claim 2 wherein said mating surfaces of said bushing form rectangular grooves.

5. The implant recited in claim 2 wherein said femoral component is configured such that a plane of symmetry is defined between said condylar portions in the anterior-posterior direction; and
said bushing has a base portion including said mating surfaces defining a central axis lying in said plane of symmetry, and an upper portion defining a central axis offset from said plane of symmetry at an acute angle.

6. The implant recited in claim 5 further comprising a femoral stem configured for insertion, in use, into the femoral canal, and wherein said upper portion of said bushing is configured for secure attachment to said femoral stem to align said femoral stem at said acute angle along the central axis of said upper portion.

7. The implant recited in claim 5 wherein said bushing defines a bore therethrough for receiving said locking member.

8. The implant recited in claim 7 wherein said locking member is a set screw.

9. The implant recited in claim 8 wherein said set screw is threaded.

10. The implant recited in claim 8 wherein said set screw further comprises a proximal surface and a distal surface.

11. The implant recited in claim 10 wherein said proximal surface is structured for receiving a tool for tightening said set screw.

12. The implant recited in claim 11 wherein said distal surface is a surface that, in use, upon the application of torque, extends beyond said base portion of said bushing and contacts said femoral component so as to form a pressure fitting against said femoral component.

13. The implant recited in claim 7 wherein said bore further defines a minor bore in said base portion for receiving said locking member and a major bore in said upper portion for receiving said femoral stem.

14. The implant recited in claim 13 wherein said minor bore is threaded.

15. The implant recited in claim 13 wherein said major bore is threaded.

16. The implant recited in claim 5 wherein the central axis of said upper portion of said bushing is offset to a degree to substantially match the valgus angle of a recipient of said implant.

17. The implant recited in claim 16 wherein the valgus angle is between 0 and about 9 degrees from said central axis.

18. The implant recited in claim 1 wherein said bushing defines a bore therethrough for receiving said locking member.

19. The implant recited in 18 wherein said bore is threaded.
